(19) United States
(12) Patent Application Publication
(10) Pub. No.: US 2024/0139685 A1
(43) Pub. Date: May 2, 2024

(54) SYSTEMS AND METHODS FOR SINGLE PASS COUNTER CURRENT DIAFILTRATION

(71) Applicants: Nikita IKON, Ridgefield, CT (US); Philip Brian NELSON, Ridgefield, CT (US); Boehringer Ingelheim International GmbH, Ingelheim am Rhein (DE)

(72) Inventors: Nikita IKON, Union City, CA (US); Philip Brian NELSON, Los Gatos, CA (US)

(21) Appl. No.: 18/280,884

(22) PCT Filed: Dec. 14, 2021
(86) PCT No.: PCT/US2021/063190

§ 371 Date: Sep. 7, 2023

Related U.S. Application Data
(60) Provisional application No. 63/157,877, filed on Mar. 8, 2021.

(51) Int. Cl. B01D 63/08; B01D 61/14; B01D 61/18; C07K 1/34

(52) U.S. Cl. CPC B01D 63/085; B01D 61/146; B01D 61/147; B01D 61/149; B01D 61/18; C07K 1/34; B01D 2313/243; B01D 2315/10; B01D 2315/16; B01D 2317/022

(57) ABSTRACT

Systems and methods for single-pass, countercurrent diafiltration of a fluid feed are provided. The systems and methods operate to reduce impact on the biologic product, reduce use of diafiltration buffer and reduce equipment size compared to traditional systems.

### RELATED APPLICATIONS

This application claims the priority benefit of U.S. Provisional Application No. 63/157,877, filed Mar. 8, 2021. The entire content of the above application is incorporated herein by reference as though fully set forth herein.

### FIELD OF THE ART

Disclosed herein are systems and methods for diafiltration of biologic products. More particularly, the diafiltration systems and methods operate to reduce impact on the biologic product, reduce use of diafiltration buffer and reduce equipment size compared to traditional systems.

### BACKGROUND

Diafiltration is a dilution process that involves removal or separation of components of a solution based on their molecular size by using micro-molecule permeable filters in order to obtain pure solution. Diafiltration is performed for buffer exchange during formulation of a biologic product to achieve appropriate buffer composition.

Buffer exchange is typically performed in systems that use a recirculation loop to and from a tangential flow filtration (TFF) membrane and a feed tank large enough to hold the entire batch volume. The TFF membrane allows components smaller than the molecular weight cut off to pass through the filter and into the permeate stream directed to waste, while larger components are retained in the retentate stream and circulated back to the feed tank. A required volume of diafiltration buffer, specified to attain a desired clearance factor, is added to the feed tank or retentate stream at the same rate that buffer is removed through the permeate to maintain a constant volume in the feed vessel.

Constant volume batch diafiltration is the most common process configuration. Commercial systems for batch diafiltration can involve several hundred square meters of membrane area housed in a skid. Increasing concerns about the cost of goods and manufacturing flexibility has led to a growing interest in the potential of continuous bioprocessing.

U.S. Pat. No. 10,183,108 to the Pall Corporation describes a diafiltration system designed for continuous buffer exchange. While these stages provide continuous buffer exchange, the system requires larger buffer volumes to achieve the same level of buffer exchange as a corresponding batch system, and the multihead pumping system is more complex.

Rucker-Pezzini et al. describe a three-stage single pass diafiltration system (see Rucker-Pezzini et al., “Single pass diafiltration integrated into a fully continuous mAb purification process.” Biotechnology and Bioengineering. 2018; 115:1949-1957).

Nambiar et al. describe using single pass tangential flow filtration (SPTFF) stages for continuous diafiltration (see Nambiar et al., “Countercurrent staged diafiltration for formulation of high value proteins.” Biotechnology and Bioengineering. 2018; 115:139-144). The individual stages were arranged in a countercurrent configuration to reduce buffer requirements.

There remains a need for improved diafiltration systems and methods which reduce the impact on the biologic product, reduce the consumption of diafiltration buffer and reduce the size of the necessary equipment.

### SUMMARY OF THE INVENTION

Disclosed herein are systems and methods for the efficient diafiltration of biologic products. The systems and methods can support continuous or batch processes as well as reusable or single use processing.

The systems disclosed herein offer distinct advantages over traditional tangential flow filtration (TFF) systems used for the diafiltration of biologic products. In particular, diafiltration carried out with the systems according to the embodiments operate such that the biologic product experiences only one pump pass compared to about 40 to about 80 pump passes in tradition TFF systems. In this way, potential impact on the biologic product is reduced.

Another advantage of the systems disclosed herein is a substantial reduction in the use of diafiltration buffer. For example, the use of diafiltration buffer can be reduced by at least about 60% compared to traditional TFF systems, achieving 8 diavolumes of diafiltration with 3 diavolumes of diafiltration buffer using a 3-stage system.

A further advantage of the systems disclosed herein is that the equipment size may be substantially reduced compared to traditional TFF systems. In some embodiments, the feed flow rate of the system is about 40 to about 80 fold lower than the feed flow rate of a traditional TFF system. In certain embodiments, the system is more easily produced with a single use product contacting flow path compared to a traditional TFF system. The size of a traditional TFF system might preclude the use of single use materials of construction.

The systems and methods described herein accomplish buffer exchange, or diafiltration, at a constant retentate concentration. The TFF cassettes used in the systems and methods each include a diafiltration channel, a flat first filtration membrane, a retentate channel, a flat second filtration membrane, and a permeate/diafiltration buffer collection channel, configured such that (i) the flat first filtration membrane delimits the diafiltration channel and the retentate channel from one another, (ii) the flat second filtration membrane delimits the retentate channel and the permeate/diafiltration buffer collection channel from one another, (iii) the diafiltration channel is fluidly connected to at least one inlet for the diafiltration medium, (iv) the retentate channel is fluidly connected to at least one inlet for the feed fluid and to at least one outlet for the retentate, and (v) the permeate/diafiltration buffer collection channel is fluidly connected to at least one outlet for the permeate/diafiltration buffer. The systems and methods described herein provide a stepwise diafiltration of a feed medium that comprises a molecule of interest (e.g., a protein or other biologic molecule). The feed medium is diafiltered against a diafiltration buffer across an appropriate membrane, with the diafiltration taking place several times (e.g., two, three, four or more times) in adjacent diafiltration cassettes. This stepwise diafiltration is characterized by a countercurrent flow of the diafiltration buffer against the feed medium. The permeate (and diafiltration buffer, if any remains) leaving the system from the first diafiltration cassette (e.g., the left-most cassette shown in FIG. 1) comprises washed-out impurities, mixture of ions etc., while the retentate leaving the system after the final diafiltration cassette (e.g., the right-most cassette shown in FIG. 1) comprises the molecule of interest exchanged into the diafiltration buffer.

In a first aspect, a system for single-pass, countercurrent diafiltration of a fluid feed is disclosed, the system comprising: two, three, four or more filtration stages; conduits to facilitate fluid communication between manifolds in the filtration stages; a conduit and at least one feed pump to provide a fluid feed to the first filtration unit; a conduit and at least one buffer pump to provide diafiltration buffer to the final filtration stage; a conduit and at least one buffer pump per filtration stage to provide permeate and diafiltration buffer from a subsequent to a prior filtration stage; and a conduit and a filtrate pump to remove permeate (and diafiltration buffer, if any remains) from the system;
wherein each filtration stage comprises:
(i) a manifold segment and (ii) one or more TFF cassettes as described herein; wherein the manifold segment comprises: a first manifold (i.e., inlet) for receiving and carrying the fluid feed or retentate into the filtration stage, a second manifold (i.e., outlet) for receiving and carrying retentate out of the filtration stage, a third manifold (i.e., inlet) for receiving and carrying permeate and diafiltration buffer through the filtration stage, and a fourth manifold (i.e., outlet) for receiving and carrying permeate and diafiltration buffer out of the filtration stage; and wherein the manifold segment is fluidly connected to the one or more TFF cassettes as described herein;
wherein the filtration stages are fluidly connected through the manifold segments to provide a flow path between filtration stages, by coupling of the second manifold in a manifold segment to the first manifold of a manifold segment in a subsequent filtration stage, such that the retentate of one stage serves as the feed for the next filtration stage, and by coupling of the fourth manifold in a manifold segment to the third manifold of a manifold segment in the prior filtration stage;
wherein the first filtration stage in the system receives the fluid feed from a conduit connected to a feed pump; and wherein the second manifold on the final (i.e., last) filtration stage is a retentate outlet that allows the retentate to leave the system and does not connect to a prior filtration stage.

In certain embodiments, the system comprises two filtration stages. In certain embodiments, the system comprises three filtration stages.

In a second aspect, a method of filtering a fluid feed using the system described herein is provided.

In a third aspect, a method of manufacturing a biologic product of interest is provided, the method comprising the steps of:
(I) cultivating a eukaryotic cell expressing the biologic product of interest in cell culture;
(II) harvesting the biologic product of interest from the cell culture in the form of a fluid feed comprising (a) the biologic product of interest and (b) one or more impurities and/or buffer components;
(III) purifying the fluid feed comprising the biologic product of interest and the one or more impurities and/or buffer components to isolate the biologic product of interest from the fluid feed; and
(IV) optionally formulating the biologic product of interest into a pharmaceutically acceptable formulation suitable for administration; and
wherein the method further comprises: (a) passing the fluid feed through a single-pass, countercurrent diafiltration system, and (b) recovering permeate (and diafiltration buffer, if any remains) and retentate from the system in separate containers; and
wherein the system is a system according to the embodiments described herein.

Additional advantages of the subject technology will become readily apparent to those skilled in this art from the following drawings and the detailed description. The drawings and description are to be regarded as illustrative in nature, and not as restrictive.

### BRIEF DESCRIPTION OF FIGURES

FIG. 1 is a diagram illustrating an exemplary single pass countercurrent diafiltration system.

FIG. 2 is a graph of the diavolumes (buffer flow rate/feed flow rate) versus tracer clearance for a system with one TFF cassette according to the embodiments.

FIG. 3 is a graph of the diafiltration efficiency of a 2-stage single pass countercurrent diafiltration system.

FIG. 4 is a graph of the natural log clearance of glucose versus diavolumes of diafiltration buffer for a 3 stage single pass counter current diafiltration system. The horizontal line (9.2 natural logs of clearance) marks the limit of detection.

### DETAILED DESCRIPTION OF THE INVENTION

In the following detailed description, numerous specific details are set forth to provide a full understanding of the subject technology. It will be apparent, however, to one of ordinary skill in the art that the subject technology may be practiced without some of these specific details. In other instances, well-known structures and techniques have not been shown in detail so as not to obscure the subject technology. Preferences and options for a given aspect, feature, embodiment, or parameter of the invention should, unless the context indicates otherwise, be regarded as having been disclosed in combination with any and all preferences and options for all other aspects, features, embodiments and parameters of the invention.

To facilitate an understanding of the present subject technology, a number of terms and phrases are defined below. Unless defined otherwise, all technical and scientific terms used herein have the same meaning as commonly understood by one of ordinary skill in the art to which this invention pertains.

### Definitions

The grammatical articles “one”, “a”, “an”, and “the”, as used herein, are intended to include “at least one” or “one or more”, unless otherwise indicated. Thus, the articles are used herein to refer to one or more than one (i.e., to at least one) of the grammatical objects of the article. By way of example, “a component” means one or more components, and thus, possibly, more than one component is contemplated and may be employed or used in an implementation of the described embodiments.

The term “about” generally refers to a slight error in a measurement, often stated as a range of values that contain the true value within a certain confidence level (usually ±1σ for 68% C.I.). The term “about” may also be described as an integer and values of ±20% of the integer.

The term “biologic product” generally refers to a product of interest created via biological processes or via the chemical or catalytic modification of an existing biologic product. Biological processes include cell culture, fermentation, metabolization, respiration, and the like. Biologic products of interest include, for example, antibodies, antibody fragments, proteins, hormones, vaccines, fragments of natural proteins (such as fragments of bacterial toxins used as vaccines, e.g., tetanus toxoid), fusion proteins or peptide conjugates (e.g., such as substage vaccines), virus-like particles (VLPs) and the like.

The term “system” refers to a single-pass, countercurrent diafiltration system. Such systems configured for operation in a single-pass mode, where the fluid passes once through the system.

The term “countercurrent” refers to backfeeding of filtrate stream into the retentate stream at a location (stage) upstream of where the specific filtrate was produced. As such, countercurrent can refer to multistage backfeeding of filtrate streams.

The term “TFF cassette” generally refers to a tangential flow filtration device, which includes a membrane suitable for separating the feed solution containing biologic products into retentate and permeate streams. A “TFF cassette” refers to a plate-and-frame structure comprising a filtration membrane and separate feed/retentate and permeate flow channels suitable for a TFF or diafiltration process. In particular, the TFF cassettes used in the systems and methods each include a diafiltration channel, a flat first filtration membrane, a retentate channel, a flat second filtration membrane, and a permeate/diafiltration buffer collection channel, arranged such that the flat first filtration membrane delimits the diafiltration channel and the retentate channel from one another, and the flat second filtration membrane delimits the retentate channel and the permeate/diafiltration buffer collection channel from one another. The diafiltration channel is fluidly connected to at least one inlet for the diafiltration medium; the retentate channel is fluidly connected to at least one inlet for the feed fluid and to at least one outlet for the retentate; and the permeate/diafiltration buffer collection channel is fluidly connected to at least one outlet for the permeate/diafiltration buffer.

The term “filtration membrane” refers to a selectively permeable membrane for separating a feed into a permeate stream and a retentate stream using a TFF or diafiltration process. Exemplary filtration membranes include, but are not limited to. Hydrosart® membranes and polyethersulfone membranes (PESU), such as those available from Sartorius. Other filtration membranes may be suitable for the systems and methods described herein and such membranes would feature: a stable polymer composition, a broad pH range, and/or a broad temperature range. Typically, the filtration membranes described herein have pore sizes in the range between about 5 kiloDalton (kD) to about 300 kD, or about 0.0012 micron to about 0.075 micron. In a particular embodiment, the filtration membranes have pore sizes in the range of about 20 kD to about 40 kD, or about 25 kD to about 35 kD. In one embodiment, the filtration membrane has a pore size, or average pore size, of about 30 kD. In one embodiment, the filtration membrane has a pore size that is suitable for processing antibodies.

The term “feed” or “feed stream” refers to the solution that is delivered to a filtration stage to be filtered. The feed that is delivered to a filtration stage for filtration can be, for example, feed from a feed container (e.g., vessel, tank) external to the system, or retentate from a preceding filtration stage in the same system.

The term “filtration” generally refers to the act of separating the feed into two streams, a permeate and a retentate.

The term “permeate” refers to that portion of the feed that has permeated through the membrane.

The term “filtrate” refers to any material (permeate and/or diafiltration buffer) that has permeated through the membrane.

The term “retentate” refers to that portion of the solution that has been retained by the membrane. In certain embodiments, no enrichment (i.e., concentration) of the retentate takes place during the exemplary diafiltration process. According to the embodiments, the buffer exchange (diafiltration) is achieved at a constant retentate concentration.

“Feed conduit” refers to a conduit for conveying a feed from a feed source (e.g., a feed container) to a filtration stage.

“Retentate conduit” refers to a conduit in a filtration assembly for carrying retentate.

“Permeate conduit” refers to a conduit in a filtration assembly for carrying permeate.

The expression “flow path” refers to a channel supporting the flow of a fluid (or liquid) through all or part of a system. The fluid can be the feed, permeate, diafiltration buffer, or retentate. For example, a flow path through the entire system from the feed inlet to the retentate outlet; a flow path within a filtration stage (e.g., a flow path through TFF cassettes and/or a manifold segment in a filtration stage); and a flow path between two or more adjacent filtration stages. The flow path can have any topology which supports the desired flow (e.g., straight, coiled, arranged in zigzag fashion). The flow path can be parallel or serial. A flow path can also refer to a path resulting in a single pass through a system.

A “filtration stage” refers to a unit in a system comprising a manifold segment and one or more TFF cassettes as described herein.

A “manifold segment” refers to a block having a plurality of manifolds (e.g., inlets and outlets), including a manifold for carrying a feed, a manifold for carrying a diafiltration buffer into the filtration stage, a manifold for carrying a retentate, and a manifold for carrying a permeate/diafiltration buffer out of the filtration stage.

The term “plurality,” when used herein to describe system units, refers to two or more units.

The expression “fluidly connected” refers to two or more components of a system (e.g., two or more manifold segments, two or more TFF cassettes, a manifold segment and one or more TFF cassettes), that are connected by one or more conduits (e.g., a feed conduit, a retentate conduit, a diafiltration buffer conduit, a permeate and diafiltration buffer conduit) such that a fluid can flow from one component to the other.

The term “processing” as used herein refers to the act of filtering a feed containing a biologic product and subsequently recovering the diafiltered product. The diafiltered product can be recovered by directing the retentate to a suitable collection vessel.

The expressions “serial processing”, “processing in series”, “serial operation” and “operation in series” refer to distributing a fluid or liquid in a system to one filtration stage at a time, such that the retentate flow of a preceding unit serves as the feed flow for a subsequent, adjacent unit.

The expressions “conversion” and “conversion per pass” are used herein to denote the fraction of the feed volume that permeates through the membrane in a pass through the flow channels, expressed as a percentage of the feed stream volume.

The term “residence time” refers to holdup volume on the feed side of the membrane divided by flow rate.

The term “concentration factor” as used herein refers to the amount that the product has been concentrated in the feed stream. This depends on both the volume concentration factor and the retention.

The term “diafiltration” or “DF” is used to mean a specialized class of filtration in which the retentate is simultaneously diluted with solvent and re-filtered, to reduce the concentration of soluble permeate components.

The term “diavolume” is a measure of the extent of washing that has been performed during a diafiltration step. It is based on the volume of diafiltration buffer introduced into the stage operation compared to the retentate volume.

The terms “downstream” or “downstream processing” generally refers to some or all of the steps necessary for capture of a biologic product from the original solution in which it was created, for purification of the biologic product away from undesired components and impurities, for filtration or deactivation of pathogens (e.g., viruses, endotoxins), and for formulation and packaging.

The term “ultrafiltration” refers to filtration used to retain biologic products and pass buffer components smaller than the molecular weight cutoff into the filtrate. Ultrafiltration membranes used to retain biologic products commonly have molecular weight cutoffs between about 5 kD and about 300 kD.

The terms “polypeptide”, “polypeptide product”, “protein” and “protein product”, are used interchangeably herein and, as is known in the art, refer to a molecule consisting of two or more amino acids, e.g., at least one chain of amino acids linked via sequential peptide bonds. In one embodiment, a “protein of interest” or a “polypeptide of interest” is a protein encoded by an exogenous nucleic acid molecule that has been transformed into a host cell, wherein the exogenous DNA determines the sequence of amino acids. In another embodiment, a “protein of interest” is a protein encoded by a nucleic acid molecule that is endogenous to the host cell.

The term “retention” as used herein refers to the fraction of a particular biologic product (e.g., protein) that is retained by the membrane. It can also be calculated as either apparent or intrinsic retention.

The term “single-use” as used herein refers to articles that are suitable for one-time use with subsequent disposal, as well as reusable articles which are used only once in the process according to the invention and are then no longer used in the process. Such articles can also be referred to as “disposable”.

The term “skid” refers to a system of components contained within a frame that allows the system to be easily transported. Individual skids can contain complete process systems or systems that carry out certain aspects of a process. Multiple skids can be combined to create larger systems or entire portable plants.

The term “tangential flow filtration or “TFF”, also known as crossflow filtration, refers to a process where the feed stream flows parallel to the membrane face. Applied pressure causes one portion of the flow stream to pass through the membrane (filtrate) while the remainder (retentate) is retained.

The term “transmembrane pressure” or “TMP” refers to the average applied pressure from the feed to the filtrate side of a membrane.

The term “antibody” as used herein broadly refers to a protein capable of recognizing and binding to a specific antigen. The term specifically includes monoclonal antibodies, polyclonal antibodies, dimers, multimers, multispecific antibodies (e.g., bispecific antibodies), antibody fragments (e.g., Fab fragment, F(ab′)₂, Fv fragment or Fc fragment from a cleaved antibody, a scFv-Fc fragment, a minibody, a diabody or a scFv) and double and single chain antibodies. The term also includes human antibodies, humanized antibodies, chimeric antibodies and antibodies specifically binding cancer antigen. Furthermore, the term includes genetically engineered derivatives of an antibody. Antibodies, fragments of antibodies and genetically engineered antibodies may be obtained by methods that are known in the art.

The term “culture” and “cell culture” and “mammalian cell culture” as used herein refer to a cell population, either surface-attached or in suspension, that is maintained or grown in medium under conditions suitable for survival and/or growth of the cell population. These terms can also refer to the cell population and the medium in which the population is suspended.

The term “impurities” as used herein refers to refers to undesired chemical or biological compounds produced during the culturing process. Impurities may include, e.g., ethyl alcohol, butyl alcohol, lactic acid, acetone ethanol, gaseous compounds, peptides, lipids, ammonia, aromatic compounds, and DNA and RNA fragments, as well as media components and break down products of the biologic products.

The term “media” or “medium” or “cell culture medium” as used herein refers to a solution containing nutrients which nourish cultured cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation.

### Diafiltration System

Disclosed herein is a system for single-pass, countercurrent diafiltration and methods of filtering a fluid feed (e.g., liquid feed or solution feed) using the system.

In one embodiment, the system and method relates to diafiltration of a fluid feed, wherein the fluid feed and diafiltration buffer flows through a single-pass diafiltration system described herein to diafilter and recover the retentate.

The diafiltration system comprises a plurality of filtration stages that are fluidly connected such that the retentate moves serially from the first to subsequent filtration stages until it reaches the last filtration stage, and also such that the permeate and diafiltration buffer flows countercurrently from later to earlier stages. Each “filtration stage” includes one or more TFF cassettes, and a manifold segment which comprises a block having a plurality of manifolds (e.g., inlets and outlets). In certain embodiments, the one or more TFF cassettes is inside, or contained by, the manifold segment. In certain embodiments, the system comprises two or more filtration stages. In certain embodiments, the system comprises three or more filtration stages. In certain embodiments, the system comprises 2, 3, or 4 stages. Generally, there is no particular limit on the number of stages that could be used in the system. However, the systems and methods are designed to achieve improved diafiltration in 2, 3 or 4 stages. In particular, the systems and methods are designed to achieve at least 8 diavolumes of diafiltration, which is commonly required for biologic products, in 2, 3 or 4 stages. In certain embodiments, the systems and methods achieve 8 diavolumes of diafiltration in 2 stages. In certain embodiments, the systems and methods achieve 8 diavolumes of diafiltration in 3 stages. In certain embodiments, the systems and methods achieve 8 diavolumes of diafiltration in 4 stages.

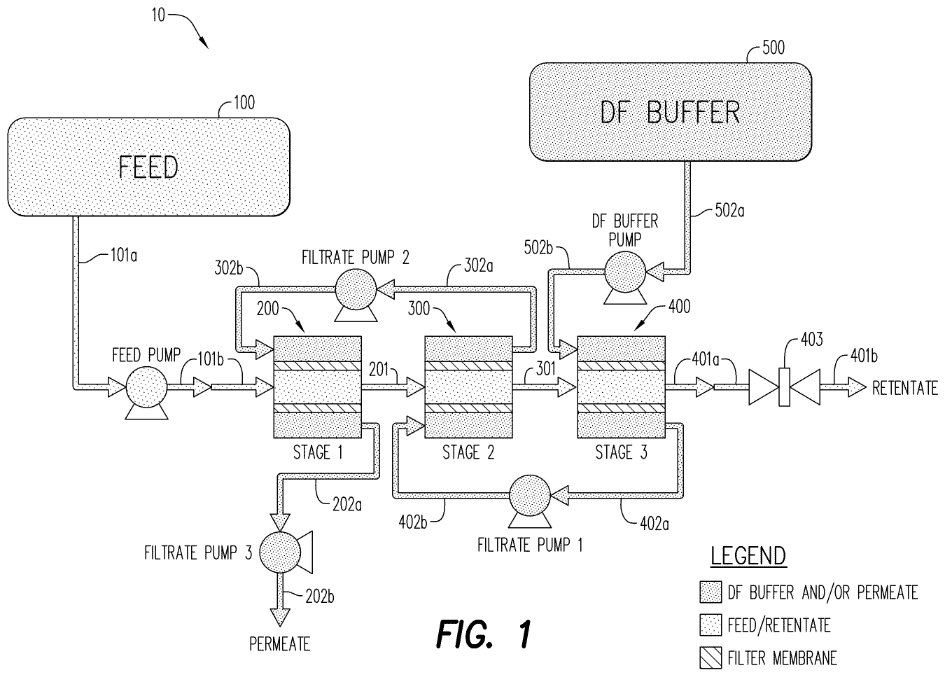

An exemplary system (10) is shown in FIG. 1. Referring to FIG. 1, fluid feed from a feed source (100) flows into a first filtration stage (“Stage 1”) via a feed conduit (101a, 101b) into a feed inlet of the first filtration stage. The fluid feed is forced through the feed conduit by a feed pump (i.e., “Feed Pump”). The feed passes through a TFF cassette (200), which separates retentate from permeate and diafiltration buffer present in the first filtration stage. The retentate flows into a subsequent (i.e., second) filtration stage via a feed conduit (201). Permeate and diafiltration buffer flow into the first filtration stage via a permeate and diafiltration buffer conduit (302a, 302b) into a permeate and diafiltration buffer inlet. The permeate and diafiltration buffer are forced through the permeate and diafiltration buffer conduit from the second filtration stage to the first filtration stage by a buffer pump (aka filtrate pump) (i.e., “Filtrate Pump 2”). The permeate (and diafiltration buffer, if any remains) flows out of the first filtration stage via an outlet on the first filtration stage. The permeate (and diafiltration buffer, if any remains) is forced through a permeate or filtrate conduit (202a, 202b) by a filtrate pump (“i.e., “Filtrate Pump 3”). The permeate (and diafiltration buffer, if any remains) may be disposed of, accordingly.

In the second filtration stage (“Stage 2”) shown in FIG. 1, the retentate from stage 1 passes through a TFF cassette (300), which separates the retentate from the permeate and diafiltration buffer present in the second filtration stage. The retentate from stage 2 flows into a subsequent (i.e., third) filtration stage via a feed conduit (301). Permeate and diafiltration buffer flow from the third filtration stage into the second filtration stage via a permeate and diafiltration buffer conduit (402a, 402b) into a permeate inlet. The permeate and diafiltration buffer are forced through the permeate and diafiltration buffer conduit from the third filtration stage to the second filtration stage by a buffer pump (i.e., “Filtrate Pump 1”).

As shown in FIG. 1, diafiltration buffer (“DF buffer”) flows from a diafiltration buffer source (500) into a final (i.e., third) filtration stage (“Stage 3”) through a diafiltration buffer conduit (502a, 502b) into a diafiltration buffer inlet on the final filtration stage. A buffer pump (i.e., “DF Buffer Pump”) forces the diafiltration buffer through the diafiltration buffer conduit into the final filtration stage. In the final filtration stage, the retentate from stage 2 passes through a TFF cassette (400), which separates the retentate from the permeate and diafiltration buffer present in the third filtration stage. The retentate from stage 3 exits the system via the retentate outlet and retentate conduit (401a, 401b). The retentate may then be stored or continue on to other downstream components to undergo other processes. An optional valve (403) can be used to regulate backpressure.

As mentioned above, each of the filtration stages comprises a manifold segment that includes a first manifold (i.e., inlet) for receiving and carrying the feed (or retentate) into the filtration stage, a second manifold (i.e., outlet) for receiving and carrying retentate out of the filtration stage, a third manifold (i.e., inlet) for receiving and carrying permeate and/or diafiltration buffer through the filtration stage, and a fourth manifold (i.e., outlet) for receiving and carrying permeate and diafiltration buffer out of the filtration stage. The filtration stages are fluidly connected through the manifold segments to provide a flow path between filtration stages, by coupling of the second manifold in a manifold segment to the first manifold of a manifold segment in a subsequent stage, such that the retentate of one stage serves as the feed for the next stage, and by coupling of the fourth manifold in a manifold segment to the third manifold of a manifold segment in the prior filtration stage. The manifold segment in each stage is also fluidly connected to one or more TFF cassettes, which may be stacked on one or both faces of the manifold segment. In addition, the system comprises a feed inlet on the first stage in the system and a retentate outlet on the last stage in the system such that the feed initially enters the system through the first filtration stage and the retentate (i.e., product) exits the system from the last filtration stage.

The systems described herein utilize countercurrent flow of the permeate and diafiltration buffer, i.e., the flow path of the permeate and diafiltration buffer moves through conduits and filtration stages in the direction of last to first filtration stage. FIG. 1 shows, by way of example, a simple three-stage system in which each stage has a single TFF cassette and each TFF cassette has a single filter cell with two membranes delimiting a single diafiltration channel, a single feed/retentate channel, and a single permeate/buffer collection channel. As will be apparent to the skilled artisan, more complex arrangements (e.g., multiple TFF cassettes per stage, multiple filter cells per TFF cassette, etc.) can also be used as long as appropriate delineation between the diafiltration channel(s), feed/retentate channel(s) and permeate/buffer collection channel(s) is maintained.

The filtration area of the cassette can be any suitable filtration area. In certain embodiments, the system has a filtration area in the range of about 0.3 to about 80 m², about 0.3 to about 3 m², or about 10 to about 80 m² per cassette. In one embodiment, the system has a filtration area in the range of about 0.3 to about 3 m² per cassette.

The fluid feed can be any liquid (e.g., a biological liquid) or solution that contains biologic products or particles (e.g., viral particles, host cell proteins) to be filtered. For example, the fluid feed can contain a biologic product (e.g., a target protein, such as a recombinant protein) and one or more impurities (e.g., non-target proteins) and/or buffer components. Typically, the systems and methods are used for buffer exchange that occurs at or near the end of a purification process for biologic products. In certain embodiments, the buffer components removed during diafiltration are not proteins.

In certain embodiments, the fluid feed is obtained from a source of the biologic product (e.g., a hybridoma or other host cell expressing a monoclonal antibody (MAb)). In a particular embodiment, the biologic product in the fluid feed is a MAb present in the elution buffer from a chromatography step. In a particular embodiment, the diafiltration buffer is the formulation buffer for the drug substance.

Operating the system in single-pass mode allows direct flow-through diafiltration of a biologic product in the absence of recirculation, which reduces overall system size through elimination of mechanical components and permits continuous operation at high conversion levels.

In general, systems of the present invention can be assembled and operated using standard, existing components (e.g., TFF system) that are well known and are commercially available. Standard TFF system components include, for example, cassette holders, conduits (e.g., tubing, piping) for feed, retentate, permeate and buffer, a housing or enclosure, valves, gaskets, a pump stage (e.g., pump stage comprising a pump housing, diaphragm and check valve) one or more reservoirs (e.g., process containers for feed, retentate, permeate and buffer) and a pressure gauge.

### TFF Cassettes

TFF cassettes suitable for use in the exemplary systems and methods comprise a plate-and-frame structure comprising filtration membranes and separate channels for feed/retentate, for permeate/diafiltration buffer flowing into a stage, and for permeate/diafiltration buffer flowing out of a stage, suitable for a TFF or diafiltration process. Generally, the TFF cassettes comprise a plurality of adjacent crossflow filtration units (i.e., filter cells), which typically consist of repeated arrays of a retentate channel for the feed fluid or the retentate to be filtered, two flat filtration membrane layers, a permeate/diafiltration buffer intake channel and a permeate/diafiltration buffer collection channel. The permeate/diafiltration buffer intake and collection channels of the filter cell are delimited from the retentate channel of the next filter cell by additional flat membrane layers. Each retentate channel is connected to an inlet for the feed fluid to be filtered and to an outlet for the retentate in a fluid conducting (communicating) manner; each permeate/diafiltration buffer intake channel is connected to an inlet for the permeate/diafiltration buffer in a fluid conducting manner; and each permeate/diafiltration buffer collection channel is connected to an outlet for the permeate/diafiltration buffer in a fluid conducting manner.

In one embodiment, the TFF cassette comprises a diafiltration channel, a flat first filtration membrane, a retentate channel, a flat second filtration membrane, and a permeate/diafiltration buffer collection channel, configured such that (i) the flat first filtration membrane delimits the diafiltration channel and the retentate channel from one another, (ii) the flat second filtration membrane delimits the retentate channel and the permeate/diafiltration buffer collection channel from one another, (iii) the diafiltration channel is fluidly connected to at least one inlet for the diafiltration medium, (iv) the retentate channel is fluidly connected to at least one inlet for the feed fluid and to at least one outlet for the retentate, and (v) the permeate/diafiltration buffer collection channel is fluidly connected to at least one outlet for the permeate/diafiltration buffer.

In certain embodiments, the first filtration membrane and the second filtration membrane have a respective pore size or a respective molecular weight cut-off; and the pore size or the molecular weight cut-off of the first filtration membrane is at least as large as the pore size or the molecular weight cut-off of the second filtration membrane.

In certain embodiments, the first filtration membrane is a microfiltration membrane or an ultrafiltration membrane, and/or the second filtration membrane is an ultrafiltration membrane.

In one embodiment, the first filtration membrane has a molecular weight cut-off (MWCO) in the range of 30 kD to 1,500 kD. The second filtration membrane has a molecular weight cutoff in the range of 5 kD to 1,500 kD, about 5 kD to about 300 kD, about 20 kD to about 40 kD, or about 25 kD to about 35 kD.

In certain embodiments, the one or more TFF cassettes comprises a plurality of stacked arrays each including a respective diafiltration channel, a respective first filtration membrane, a respective retentate channel, a respective second filtration membrane, and a respective permeate/diafiltration buffer collection channel, such that the stacked arrays are combined to form a filter cassette.

In certain embodiments, a free volume of the diafiltration channel and/or the retentate channel decreases in a flow direction from the inlet for the feed fluid to the outlet for the retentate.

In certain embodiments, a plurality of layers of textile materials are arranged one above another in the retentate channel such that the free volume of the retentate channel decreases in the flow direction.

In certain embodiments, the filtration membranes of the one or more TFF cassettes are stabilized cellulose derivative polymer filtration membranes. In certain embodiments, the TFF cassettes each comprise one or more stabilized cellulose derivative polymer filtration membranes, for example a stabilized cellulose derivative polymer membrane that has been optimized for the biotechnological and pharmaceutical industries. In certain embodiments, the stabilized cellulose derivative polymer membrane has a broad pH and temperature range and is hydrophilic. In certain embodiments, the stabilized cellulose derivative polymer membrane resists protein binding and fouling. In certain embodiments, the stabilized cellulose derivative polymer membrane can be sterilized by either steam or autoclaving. In certain embodiments, the stabilized cellulose derivative polymer membrane can be regenerated or depyrogenated using NaOH, with or without heat. In certain embodiments, the one or more TFF cassettes can be used to remove the following from fluid feeds: mammalian cells, such as CHO and BHK; Bacteria, such as E. coli and Pasteurella C. diphtheria; yeasts; and cell lysate products. In certain embodiments, the filtration membrane, or polymer membrane, in the TFF cassette has a pore size in the range of about 5 kD to about 300 kD, or about 0.0012 micron to about 0.075 micron. In a particular embodiment, the filtration membranes have pore sizes in the range of about 20 kD to about 40 kD, or about 25 kD to about 35 kD. In one embodiment, the filtration membrane has a pore size of about 30 kD. In one embodiment, the filtration membrane has a pore size that is suitable for processing antibodies.

In certain embodiments, the filtration membrane, or polymer membrane, in the TFF cassette has a membrane area of about 0.3 to about 3.0 m². In certain embodiments, the filtration membrane, or polymer membrane, in the TFF cassette has a filtration area of about 10 to about 80 m².

In certain embodiments, the filtration membranes of the one or more TFF cassettes are polyethersulfone (PESU) filtration membranes. In certain embodiments, the TFF cassettes each comprise one or more PESU polymer filtration membranes, for example a PESU polymer membrane that has been optimized for the biotechnological and pharmaceutical industries. In certain embodiments, the PESU membrane has a broad pH range, broad temperature range and is hydrophilic. In certain embodiments, the PESU membrane resists protein binding and fouling. In certain embodiments, the PESU membrane can be sterilized by either steam or autoclaving. In certain embodiments, the PESU membrane can be regenerated or depyrogenated using NaOH, with or without heat. In certain embodiments, the PESU filtration membrane in the TFF cassette has a pore size in the range about 0.05 μm to about 0.2 μm, or about 0.1 μm to about 0.2 μm.

In certain embodiments, each diafiltration cassette is of the same basic design. In other embodiments, a combination of different diafiltration cassettes (e.g., different membrane materials, different pore sizes, etc.) can be used.

In one embodiment, the system for single-pass, countercurrent diafiltration of a fluid feed comprises: two or more filtration stages; conduits to facilitate fluid communication between manifolds in the filtration stages; a conduit and at least one feed pump to provide a fluid feed to the first filtration unit; a conduit and at least one buffer pump to provide diafiltration buffer to the final filtration stage; a conduit and at least one buffer pump per filtration stage to provide permeate and diafiltration buffer from a subsequent to a prior filtration stage; and a conduit and a filtrate pump to remove permeate (and diafiltration buffer, if any remains) from the system;
wherein each filtration stage comprises:
(i) a manifold segment and (ii) one or more TFF cassettes; wherein the manifold segment comprises: a first manifold (i.e., inlet) for receiving and carrying the fluid feed or retentate into the filtration stage, a second manifold (i.e., outlet) for receiving and carrying retentate out of the filtration stage, a third manifold (i.e., inlet) for receiving and carrying permeate and diafiltration buffer through the filtration stage, and a fourth manifold (i.e., outlet) for receiving and carrying permeate and diafiltration buffer out of the filtration stage; and wherein the manifold segment is fluidly connected to the one or more TFF cassettes;
wherein the filtration stages are fluidly connected through the manifold segments to provide a flow path between filtration stages, by coupling of the second manifold in a manifold segment to the first manifold of a manifold segment in a subsequent filtration stage, such that the retentate of one stage serves as the feed for the next filtration stage, and by coupling of the fourth manifold in a manifold segment to the third manifold of a manifold segment in the prior filtration stage;
wherein the first filtration stage in the system receives the fluid feed from a conduit connected to a feed pump; and wherein the second manifold on the final (i.e., last) filtration stage is a retentate outlet that allows the retentate to leave the system and does not connect to a prior filtration stage.

In certain embodiments, the manifold segment in each stage is also fluidly connected to one or more TFF cassettes. In certain embodiments, the one or more TFF cassettes are inside the manifold segment and are stacked on one or both faces of the manifold segment.

Each manifold segment has a manifold structure, or arrangement, that permits the segment to be fluidly connected to manifold segments in adjacent filtration stages. The manifold segments are connected in a manner that promotes a serial flow path from manifold segment to manifold segment. For example, adjacent manifold segments are arranged such that the first manifold in each manifold segment is connected to the second manifold of an adjacent manifold segment. As a result of this arrangement, the retentate of one stage (which exits the stage through the second manifold in the manifold segment) serves as the feed for the next stage (which is received in the first manifold of the manifold segment). The manifolds in the manifold segment provide a separate path for discharging the permeate and diafiltration buffer from the filtration stages and for receiving the permeate and diafiltration buffer in the prior filtration stage in the series (i.e., countercurrent flow of the permeate and diafiltration buffer).

In certain embodiments, parallel flow between adjacent manifold segments is prevented using seals or valves (e.g., sanitary valves) to facilitate a serial flow path between stages. For example, seals or valves can be positioned in the manifolds that carry feed and retentate to block fluid from flowing in a parallel fashion into adjacent manifold segments. The use of seals or valves to prevent parallel flow is particularly desirable when the manifold segments are fully bored, such that the first, second and third manifolds each extend completely through the manifold segment.

Suitable seals (e.g., mechanical seals) for placement in manifolds include, but are not limited to, rings (e.g., o-rings, metal rings), molding, packing, sealants and gaskets. Preferably, the seal is a gasket, such as, for example, a gasket that closes off an opening or a gasket having a length sufficient to closes off any dead volume between the opening and a first passage in a manifold. Preferably, the gasket is flexible and sanitary (e.g., a gasket that is non-shedding, cleanable, sanitizable, and has low extractables). The gasket can include an elastomeric material or metal (e.g., a metal foil).

The use of valves instead of seals provides greater operational flexibility by permitting parallel flow between manifold segments when the valves are open, and serial flow when the valves are closed. Suitable valves for use in manifolds include, for example, pinch valves (e.g., diaphragm valve). Preferably, the valve is low shear and sanitary (e.g., compatible, non-toxic, sanitizable, non-shedding). As used herein, a “sanitary valve” is a valve that can maintain a sterile connection regardless of whether the valve is open or closed. Typically, a sanitary valve will be compatible, non-toxic, sanitizable and non-shedding.

The manifold segment in each filtration stage is also fluidly connected to one or more TFF cassettes as described herein. For example, the manifold segment can be fluidly connected to TFF cassettes through a flow channel that extends from the first, or feed, manifold in the manifold segment through the plurality of TFF cassettes, and a retentate flow channel that extends through the plurality of TFF cassettes back to the second, or retentate, channel in the manifold segment.

The TFF cassettes can be located (e.g., stacked) on one or both faces of the manifold segment. Typically, each filtration stage can accommodate up to about 10 m 2 of filtration membrane area on each face of the manifold segment for a total of about 20 m 2 of area per filtration stage. Generally, the number of cassettes that can be stacked on each side of the manifold segment depends on the membrane area of the particular cassette. In certain embodiments, the filtration stages in a system each contain the same number and arrangement of TFF cassettes.

In one embodiment, TFF cassettes (e.g., about 1, 2, 3, 4, 5, 6, 7, 8, 9, 10 or more TFF cassettes) are located on both faces of the manifold segment. In another embodiment, TFF cassettes (e.g., about 1, 2, 3, 4, 5, 6, 7, 8, 9, 10 or more TFF cassettes) are located on only one face of the manifold segment. When TFF cassettes are located on both faces of a manifold segment, the number of TFF cassettes on each face of the manifold segment can differ or be the same. In certain embodiments, the total number of TFF cassettes on each face of the manifold segment is identical.

In certain embodiments, the systems can include one or more filtration stages with cassettes that are configured for processing in parallel, and one or more filtration stages with cassettes that are configured for processing in series (e.g., using valves, gaskets or diverter plates). Preferably, the filtration stages with cassettes that are configured for processing in parallel precede the filtration stages having cassettes that are configured for processing in series in the system. In a particular embodiment, all of the filtration stages in a system have cassettes that are configured for processing in parallel, except for the last, or final, filtration stage, which has cassettes arranged for processing in series.

An end plate or cassette holder is generally used to hold, or seal, the TFF cassettes in the filtration stage. The end plates and cassette holders can be fitted for use with particular cassettes.

The systems of the present invention typically include a feed inlet and retentate outlet. In general, the feed inlet is positioned on the first filtration stage in the system, and is connected on one end to a conduit (e.g., pipe, tube) that is connected to the feed tank and is connected on the other end to the first manifold in the manifold segment in the first stage to receive feed into the system. The retentate outlet is typically positioned on the last, or final, filtration stage in the system, and is connected on one end to the second manifold in the manifold segment in the last stage and is connected on the other end to a conduit (e.g., pipe, channel) that is connected to a retentate container.

The systems described herein can further contain one or more additional components useful for performing diafiltration processes including, but not limited to, the following, examples of which are known in the art: one or more sampling ports, a T-line (e.g., for in-line buffer addition), a pressure sensor, a diaphragm for a pressure sensor, a valve sensor to indicate whether any valves in the system are open or closed, and a flow meter. In a particular embodiment, the system includes a sampling port (e.g., sanitary sampling port) at one or more locations in the system. For example, sampling ports can be included at the end of the retentate line, the permeate/diafiltration buffer line, or both. Typically, the sampling port will be located on the manifold segment in a filtration stage. In one embodiment, the system lacks diverter plates.

In some embodiments, one or more components of the system can be disposable.

In certain embodiments, the system comprises flow meters on the diafiltration buffer lines. In certain embodiments, the system comprises flow meters on the filtrate lines. In certain embodiments, the system comprises flow meters on the product feed lines. In certain embodiments, the system comprises flow meters on the retentate lines. In certain embodiments, the system comprises automation to control the relative flow rates between product and diafiltration buffer and/or filtrate.

Generally, all of the buffer (or filtrate) pumps are controlled (either mechanically or through automation) to operate at the same flow rate. This can be achieved at the laboratory scale by using a multi-headed peristaltic pump to achieve the same flow rate through each pump while maintaining the ability to vary the overall flow rate.

### Methods

Disclosed herein is a method for filtering a fluid feed using the single-pass, countercurrent diafiltration system described herein.

In one embodiment, the method relates to diafiltration of a fluid feed, wherein the fluid feed and diafiltration buffer flow through a single-pass diafiltration system described herein to separate and recover retentate and permeate portions of the fluid feed.

In one embodiment, the method comprises passing a fluid feed through a single-pass, countercurrent diafiltration system, and recovering permeate (and diafiltration buffer, if any remains) and retentate from the system in separate containers. In certain embodiments, the permeate/buffer and retentate are not recirculated through the system (i.e., the method occurs in a single pass through the system).

The methods described herein comprise performing diafiltration (e.g., to remove or lower the concentration of salts or solvents in the fluid feed, or to accomplish buffer exchange). In certain embodiments, the diafiltration is performed by concentrating the fluid feed to reduce the diafiltration volume and then restoring the feed to its starting volume by adding diafiltration solution, a process which is known in the art as discontinuous, or batch, diafiltration. In another embodiment, diafiltration is performed by adding the diafiltrate solution to retentate to increase the diafiltration volume followed by concentrating the sample to restore it to its original volume. In yet another embodiment, the diafiltration is performed by adding the diafiltration solution to unfiltered feed at the same rate that permeate is removed from the system, a process which is known in the art as continuous, or constant-volume, diafiltration. Suitable diafiltration solutions are well known and include, for example, water and various aqueous diafiltration buffer solutions.

To perform diafiltration, the system can include a reservoir or container for diafiltration solution and one or more conduits for carrying diafiltration solution from the diafiltration solution container to the final filtration stage.

To avoid extremes of concentration and in-line dilution as part of the diafiltration process it is preferred to pump the diafiltrate into the filtration assembly so as to maintain the flow in the retentate section to the same flow as in the initial feed. This requires matching the rate of diafiltrate buffer addition with the rate of permeate removal. Each pump will have closely-matched pumping rates so this process will be balanced and maintain efficient buffer exchange.

In certain embodiments, a two-stage system can be used to achieve a diafiltration equivalent of at least 8 diavolumes used in batch TFF.

Methods of manufacturing or producing of the biologic product of interest known in the art may be used in combination with the systems and methods of filtering a fluid feed described herein. For example, a person of skill in the art knows how to manufacture or produce biologic products, such as recombinant proteins, using fermentation. In certain embodiments, the production of biologic product of interest comprises cultivating a eukaryotic cell expressing the biologic product of interest in cell culture. Cultivating the eukaryotic cell expressing the biologic product of interest in cell culture may comprise maintaining the eukaryotic cells in a suitable medium and under conditions that allow growth and/or protein production/expression. The biologic product of interest may be produced by fed-batch or continuous cell culture. Thus, the eukaryotic cells may be cultivated in a fed-batch or continuous cell culture, preferably in a continuous cell culture.

In certain embodiments, the eukaryotic host cells are yeast cells. In one embodiment, the eukaryotic host cell is a mammalian cell. Mammalian cells as used herein are mammalian cells lines suitable for the production of a secreted recombinant therapeutic protein and may hence also be referred to as “host cells”. In certain embodiments, the mammalian cells are rodent cells such as hamster cells. The mammalian cells are isolated cells or cell lines. In certain embodiments, the mammalian cells are transformed and/or immortalized cell lines. In certain embodiments, the mammalian cells are adapted to serial passages in cell culture and do not include primary non-transformed cells or cells that are part of an organ structure. In certain embodiments, the mammalian cells are BHK21, BHK TK-, Jurkat cells, 293 cells, HeLa cells, CV-1 cells, 3T3 cells, CHO, CHO-K1, CHO-DXB11 (also referred to as CHO-DUKX or DuxB11), CHO-S cells or CHO-DG44 cells or the derivatives/progenies of any of such cell line. In certain embodiments, the mammalian cells are CHO cells, such as CHO-DG44, CHO-K1 and BHK21, and even more preferred are CHO-DG44 and CHO-K1 cells. In certain embodiments, the mammalian cells are CHO-DG44 cells. Glutamine synthetase (GS)-deficient derivatives of the mammalian cell, particularly of the CHO-DG44 and CHO-K1 cell, are also encompassed. In one embodiment, the mammalian cell is a Chinese hamster ovary (CHO) cell, for example a CHO-DG44 cell, a CHO-K1 cell, a CHO DXB11 cell, a CHO-S cell, a CHO GS deficient cell or a derivative thereof. In certain embodiments, the host cell may further comprise one or more expression cassette(s) encoding a heterologous protein, such as a therapeutic protein, for example a recombinant secreted therapeutic protein. In certain embodiments, the host cells may also be murine cells such as murine myeloma cells, such as NS0 and Sp2/0 cells or the derivatives/progenies of any of such cell line.

The expression of the biologic product of interest or recombinant protein occurs in a cell comprising a DNA sequence coding for the biologic product of interest or recombinant protein, which is transcribed and translated into the protein sequence including post-translational modifications to produce the biologic product of interest or recombination protein in cell culture.

Disclosed herein is a method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising (a) the biologic product of interest and (b) one or more impurities and/or buffer components;
(III) purifying the fluid feed comprising the biologic product of interest and the one or more impurities and/or buffer components to isolate the biologic product of interest from the fluid feed; and
(IV) optionally formulating the biologic product of interest into a pharmaceutically acceptable formulation suitable for administration; and
wherein the method further comprises: (a) passing the fluid feed through a single-pass, countercurrent diafiltration system, and (b) recovering permeate (and diafiltration buffer, if any remains) and retentate from the system in separate containers;
wherein the system is a system according to the embodiments described herein.

In certain embodiments, the biologic product of interest is a recombinant protein. In certain embodiments, the step of cultivating a eukaryotic cell expressing the biologic product of interest in cell culture occurs in a fed-batch cell culture. In certain embodiments, wherein the step of cultivating a eukaryotic cell expressing the biologic product of interest in cell culture occurs in a continuous cell culture.

The following examples are presented for illustrative purposes only and are not intended to be limiting.

### EXAMPLES

### Example 1

The flow of the multiple buffer (or filtrate) pumps was controlled using a multi-headed peristaltic pump to achieve the same flow rate through each pump while maintaining the ability to vary the overall flow rate. Laboratory data was generated using 0.33 m² Sartopure® 30 kd Hydrosart® TFF cassettes and antibody solution from Boehringer Ingelheim Fremont Incorporated (BIFI). A single cassette was run across a range of flow rates. Table 1 shows the corresponding flow rates for the feed, retentate, permeate and diafiltration buffer for each experiment.

TABLE 1
Single Cassette Flow Rates

| Target | Inlet | Outlet | Feed | Retentate | Permeate | Buffer | Inlet Ratio/ |
|---|---|---|---|---|---|---|---|
| DF Ratio | Ratio | Ratio | (mL/min) | (mL/min) | (mL/min) | (mL/min) | Outlet Ratio |
| 1.5 | 1.255 | 1.277 | 47 | 47 | 60 | 59 | 0.983 |
| 2 | 1.935 | 1.735 | 46 | 49 | 85 | 89 | 1.115 |
| 2.5 | 2.353 | 2.250 | 51 | 56 | 126 | 120 | 1.046 |
| 3 | 2.860 | 2.574 | 50 | 54 | 139 | 143 | 1.111 |
| 3.5 | 3.056 | 2.780 | 54 | 59 | 164 | 165 | 1.099 |
| 4 | 4.103 | 3.548 | 29 | 31 | 110 | 119 | 1.156 |
| 4.5 | 4.500 | 4.500 | 30 | 30 | 135 | 135 | 1.000 |
| 5 | 4.028 | 3.919 | 36 | 37 | 145 | 145 | 1.028 |

The feed was an antibody solution of monoclonal antibody at about 6 g/L and 153 g/L glucose. The glucose concentration of the retentate samples was measured using GLC3D and GLC3B methods for Cedex cell counter Bio HT Glucose Test. Table 2 shows the relationship between dilution factor of glucose in antibody solution and glucose concentration determination by the Bio HT Glucose Test method.

TABLE 2
Dilution of Glucose in 6 mg/mL Monoclonal Antibody Solution

| | | Spiked | DF | | | | | |
|---|---|---|---|---|---|---|---|---|
| Label/ | | Protein | Buffer | Expected Concentration | | Actual Concentration | | % |
| Sample | Dilution | (μL) | (μL) | (g/L) | (mmol/L) | (g/L) | (mmol/L) | difference |
| A/0 DV | 1.00 | 15,000 | 0 | 153.32 | 851.07 | 153.32 | 851.07 | 0 |
| B/1 DV | 2.73 | 5,500 | 9,500 | 56.22 | 312.06 | 53.29 | 295.81 | 5 |
| C/2 DV | 7.50 | 2,000 | 13,000 | 20.44 | 113.48 | 16.77 | 93.09 | 20 |
| D/3 DV | 20.00 | 750 | 14,250 | 7.67 | 42.55 | 7.20 | 39.97 | 6 |
| E/4 DV | 50.00 | 300 | 14,700 | 3.07 | 17.02 | 2.94 | 16.29 | 4 |
| F/5 DV | 150.00 | 100 | 14,900 | 1.02 | 5.67 | 0.99 | 5.47 | 4 |
| G/6 DV | 376.00 | 40 | 15,000 | 0.41 | 2.26 | 0.42 | 2.30 | −2 |
| H/7 DV | 1,001.00 | 15 | 15,000 | 0.15 | 0.85 | 0.15 | 0.83 | 2 |
| I/8 DV | 3,001.00 | 5 | 15,000 | 0.05 | 0.28 | 0.06 | 0.33 | −16 |
| J/9 DV | 7,501.00 | 2 | 15,000 | 0.02 | 0.11 | 0.02 | 0.11 | 2 |
| K/10 DV | 15,001.00 | 1 | 15,000 | 0.01 | 0.06 | 0.00 | 0.00 | 200 |

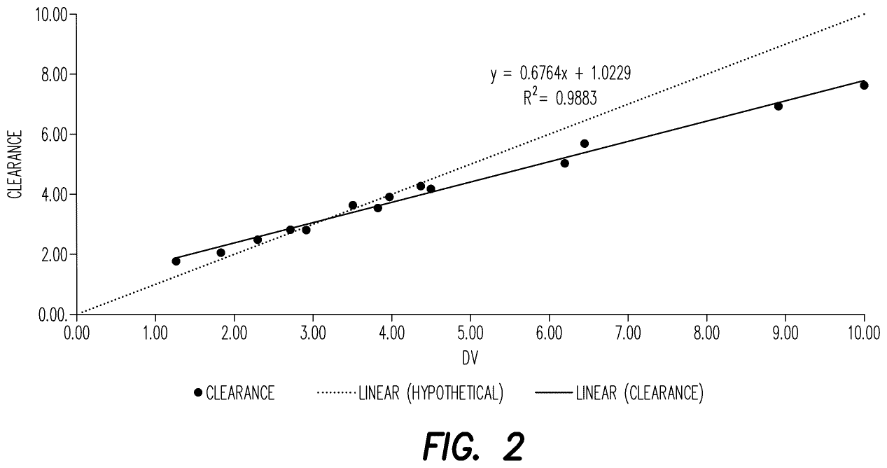

The data supports that the test method can accurately measure glucose concentrations equivalent to a diafiltration with 9 diavolumes of diafiltration buffer when starting with a glucose concentration of 153 g/L. FIG. 2 shows the relationship between ratio of diafiltration buffer flow rate and the product feed flow rate (the feed and retentate flow rates are the same) and the clearance of a tracer from the retentate stream. FIG. 2 shows the expected clearance on glucose expressed as a natural log reduction of glucose concentration versus the number of diavolumes of diafiltration buffer used (df buffer flow rate/feed flow rate). The one-to-one linear relationship shown is expected if there is an even introduction of df buffer along the length of the membrane and there is perfect mixing of df buffer and retentate. The experimental data shows that clearance is higher than these expected values when the df buffer flow rate is less than about 3× the feed flow rate and the clearance is lower than the expected values when the df buffer flow rate to feed flow rate ratio is higher than about 3.5:1 when operated at the experimental flow rates (see Table 1). Based on the data captured in FIG. 2, a system with three stages would be able to operate at a df buffer flow rate to feed flow rate ratio of 3:1 and achieve greater than 8 natural logs of clearance utilizing countercurrent flow. At this flow rate ratio and normalized flow rates similar to those in Table 1, the concentration in the retentate should remain constant throughout the operation.

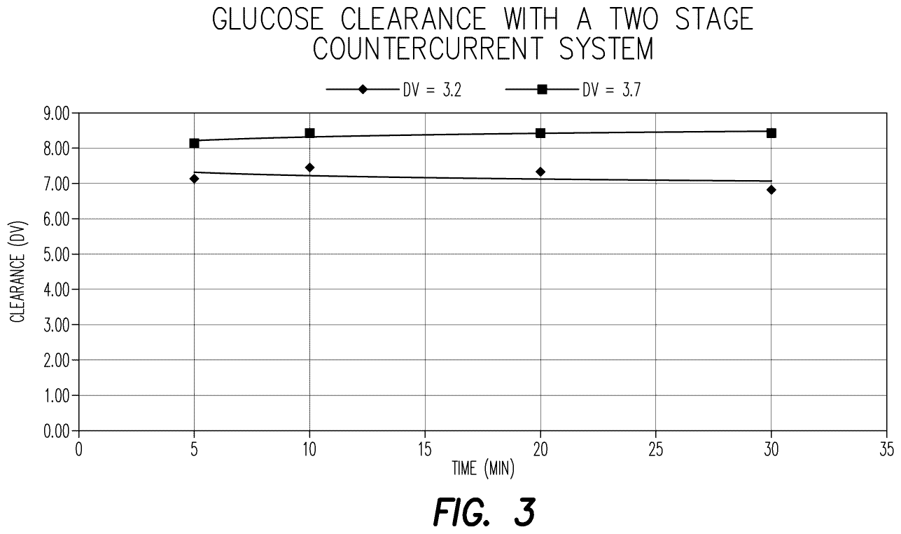

The experimental data in FIG. 3 shows that more than 8 natural logs of diafiltration (glucose reduction) can be achieved by using a less than 4:1 ratio of df buffer flow rate to feed (or retentate) flow rate in a two stage system. The experiment with a flow rate ratio of df buffer flow to retentate flow of 3.2:1 was performed by starting the flow of the df buffer first and then starting the flow on the retentate side. The experiment with a flow rate ratio of df buffer flow to retentate flow of 3.7:1 was performed by starting flow on the retentate side first and then beginning df buffer flow. The clearance is lower initially by starting up the system this way than by fully priming the system with df buffer and starting df buffer flow first. In both cases, the system will eventually reach the same steady state over time. The final clearance achieved for the 3.7 df buffer flow to retentate flow ratio in FIG. 3 represents a minimum clearance compared to actual antibody production processing conditions that would begin the operation with df buffer priming and flow. The data shows that a more than 8 diavolume diafiltration can be achieved with a counter current flow of less than 4:1 (df buffer/feed) in a two stage system. For an 8 diavolume diafiltration operation, common in biologic product manufacturing, this would reduce the diafiltration buffer used by 50%. Table 3 compares laboratory data from a 2 stage system with calculated values for a traditional TFF system diafiltering the same amount of a monoclonal antibody product in the same amount of time. The data demonstrates the efficiency gains of a multistage countercurrent system. In particular, the data supports an about 40- to about 80-fold reduction in the pump passes for the exemplary single pass countercurrent diafiltration system.

TABLE 3
Sizing Comparison Between a Two-Stage Counter
Current System and Traditional TFF

| | Exemplary System | Traditional TFF |
|---|---|---|
| Pump Passes | 1 | 40 to 80 |
| Flow Rate | 1X | 100X to 200X |
| DF Buffer Requirement | 4 diavolumes | 8 diavolumes |

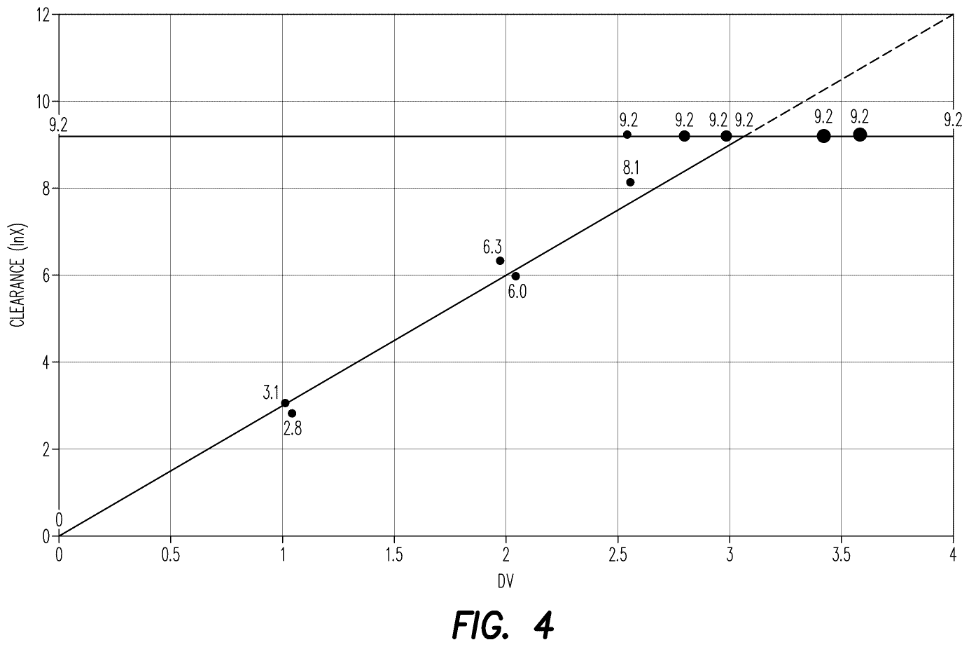

The experimental data in FIG. 4 shows that more than 8 natural logs of diafiltration (glucose reduction) can be achieved by using a less than 3:1 ratio of df buffer flow rate to feed (or retentate) flow rate in a three stage system. For an 8 diavolume diafiltration operation, common in biologic product manufacturing, this would reduce the diafiltration buffer used by 63%. Table 4 compares laboratory data from a 3 stage system with calculated values for a traditional TFF system diafiltering the same amount of a monoclonal antibody product in the same amount of time. The data demonstrates the efficiency gains of a multistage countercurrent system. In particular, the data supports an about 40- to about 80-fold reduction in the pump passes for the exemplary single pass countercurrent diafiltration system.

TABLE 4
Sizing Comparison Between a Two-Stage Counter
Current System and Traditional TFF

| | Exemplary System | Traditional TFF |
|---|---|---|
| Pump Passes | 1 | 40 to 80 |
| Flow Rate | 1X | 100X to 200X |
| DF Buffer Requirement | 3 diavolumes | 8 diavolumes |

In the preceding specification, various embodiments have been described with reference to the examples. It will, however, be evident that various modifications and changes may be made thereto, and additional embodiments may be implemented, without departing from the broader scope of the exemplary embodiments as set forth in the claims that follow. The specification and drawings are accordingly to be regarded in an illustrative rather than restrictive sense.

1. A system for single-pass, countercurrent diafiltration of a fluid feed comprising: two or more filtration stages; conduits to facilitate fluid communication between manifolds in the filtration stages; a conduit and at least one feed pump to provide a fluid feed to the first filtration stage; a conduit and at least one buffer pump to provide diafiltration buffer to the final filtration stage; a conduit and at least one buffer pump per filtration stage to provide permeate and diafiltration buffer from a subsequent to a prior filtration stage; and a conduit and a filtrate pump to remove permeate (and diafiltration buffer, if any remains) from the system;
wherein each filtration stage comprises:
(i) a manifold segment and (ii) one or more tangential flow filtration (“TFF”) cassettes; wherein the manifold segment comprises: a first manifold (i.e., inlet) for receiving and carrying the fluid feed or retentate into the filtration stage, a second manifold (i.e., outlet) for receiving and carrying retentate out of the filtration stage, a third manifold (i.e., inlet) for receiving and carrying permeate and diafiltration buffer through the filtration stage, and a fourth manifold (i.e., outlet) for receiving and carrying permeate and diafiltration buffer out of the filtration stage; and wherein the manifold segment is fluidly connected to the one or more TFF cassettes;
wherein the filtration stages are fluidly connected through the manifold segments to provide a flow path between filtration stages, by coupling of the second manifold in a manifold segment to the first manifold of a manifold segment in a subsequent filtration stage, such that the retentate of one stage serves as the feed for the next filtration stage, and by coupling of the fourth manifold in a manifold segment to the third manifold of a manifold segment in the prior filtration stage;
wherein the first filtration stage in the system receives the fluid feed from a conduit connected to a feed pump; and wherein the second manifold on the final (i.e., last) filtration stage is a retentate outlet that allows the retentate to leave the system and does not connect to a prior filtration stage; and
wherein the TFF cassettes comprise a diafiltration channel, a flat first filtration membrane, a retentate channel, a flat second filtration membrane, and a permeate/diafiltration buffer collection channel, configured such that (i) the flat first filtration membrane delimits the diafiltration channel and the retentate channel from one another, (ii) the flat second filtration membrane delimits the retentate channel and the permeate/diafiltration buffer collection channel from one another, (iii) the diafiltration channel is fluidly connected to at least one inlet for the diafiltration medium, (iv) the retentate channel is fluidly connected to at least one inlet for the feed fluid and to at least one outlet for the retentate, and (v) the permeate/diafiltration buffer collection channel is fluidly connected to at least one outlet for the permeate/diafiltration buffer.

2. The system of claim 1, wherein exactly two filtration stages are present in the system.

3. The system of claim 1, wherein the system comprises three filtration stages.

4. The system of claim 1, wherein the first filtration membrane has a molecular weight cut-off in the range of 30 kD to 1,500 kD.

5. The system of claim 1, wherein the second filtration membrane has a molecular weight cutoff in the range of 5 kD to 1,500 kD.

6. A method for filtering a fluid feed, the method comprising: (i) passing a fluid feed through a single-pass, countercurrent diafiltration system of claim 1, and (ii) recovering permeate and retentate from the system in separate containers.

7. The method of claim 6, wherein the method can be used to achieve a diafiltration equivalent of at least 8 diavolumes.

8. The method of claim 6, wherein exactly two filtration stages are present in the system.

9. The method of claim 6, wherein the system comprises three filtration stages.

10. The method of claim 6, wherein the fluid feed comprises a biologic product and one or more impurities or buffer components.

11. The method of claim 6, wherein the method occurs at or near the end of a purification process for a biologic products.

12. The method of claim 10, wherein the fluid feed is obtained from a source of the biologic product.

13. The method of claim 10, wherein the biologic product in the fluid feed is a monoclonal antibody present in the elution buffer from a chromatography step.

14. A method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising (a) the biologic product of interest and (b) one or more impurities and/or buffer components;
(III) purifying the fluid feed comprising the biologic product of interest and the one or more impurities and/or buffer components to isolate the biologic product of interest from the fluid feed; and
(IV) optionally formulating the biologic product of interest into a pharmaceutically acceptable formulation suitable for administration; and
wherein the method further comprises:
(a) passing the fluid feed through a single-pass, countercurrent diafiltration system, and (b) recovering permeate (and diafiltration buffer, if any remains) and retentate from the system in separate containers;
wherein the single-pass, countercurrent diafiltration system comprises: two or more filtration stages; conduits to facilitate fluid communication between manifolds in the filtration stages; a conduit and at least one feed pump to provide the fluid feed to the first filtration stage; a conduit and at least one buffer pump to provide diafiltration buffer to the final filtration stage; a conduit and at least one buffer pump per filtration stage to provide permeate and diafiltration buffer from a subsequent to a prior filtration stage; and a conduit and a filtrate pump to remove permeate (and diafiltration buffer, if any remains) from the system;
wherein each filtration stage comprises:
(i) a manifold segment and (ii) one or more tangential flow filtration (“TFF”) cassettes; wherein the manifold segment comprises: a first manifold (i.e., inlet) for receiving and carrying the fluid feed or retentate into the filtration stage, a second manifold (i.e., outlet) for receiving and carrying retentate out of the filtration stage, a third manifold (i.e., inlet) for receiving and carrying permeate and diafiltration buffer through the filtration stage, and a fourth manifold (i.e., outlet) for receiving and carrying permeate and diafiltration buffer out of the filtration stage; and wherein the manifold segment is fluidly connected to the one or more TFF cassettes;
wherein the filtration stages are fluidly connected through the manifold segments to provide a flow path between filtration stages, by coupling of the second manifold in a manifold segment to the first manifold of a manifold segment in a subsequent filtration stage, such that the retentate of one stage serves as the feed for the next filtration stage, and by coupling of the fourth manifold in a manifold segment to the third manifold of a manifold segment in the prior filtration stage;
wherein the first filtration stage in the system receives the fluid feed from a conduit connected to a feed pump; and wherein the second manifold on the final (i.e., last) filtration stage is a retentate outlet that allows the retentate to leave the system and does not connect to a prior filtration stage; and
wherein the TFF cassettes comprise a diafiltration channel, a flat first filtration membrane, a retentate channel, a flat second filtration membrane, and a permeate/diafiltration buffer collection channel, configured such that (i) the flat first filtration membrane delimits the diafiltration channel and the retentate channel from one another, (ii) the flat second filtration membrane delimits the retentate channel and the permeate/diafiltration buffer collection channel from one another, (iii) the diafiltration channel is fluidly connected to at least one inlet for the diafiltration medium, (iv) the retentate channel is fluidly connected to at least one inlet for the feed fluid and to at least one outlet for the retentate, and (v) the permeate/diafiltration buffer collection channel is fluidly connected to at least one outlet for the permeate/diafiltration buffer.

15. The method of claim 14, wherein the biologic product of interest is a recombinant protein.

16. The method of claim 14, wherein the step of cultivating a eukaryotic cell expressing the biologic product of interest in cell culture occurs in a fed-batch cell culture.

17. The method of claim 14, wherein the step of cultivating a eukaryotic cell expressing the biologic product of interest in cell culture occurs in a continuous cell culture.

18. The method of claim 14, wherein steps (a) and (b) can be used to achieve a diafiltration equivalent of at least 8 diavolumes.

19. The method of claim 14, wherein exactly two filtration stages are present in the system.

20. The method of claim 14, wherein the system comprises three filtration stages.
